(19) United States
(12) Patent Application Publication
(10) Pub. No.: US 2023/0316521 A1
(43) Pub. Date: Oct. 5, 2023

(54) IMAGE-BASED DETERMINATION OF A PROPERTY OF A FLUORESCING SUBSTANCE

(71) Applicant: Intuitive Surgical Operations, Inc., Sunnyvale, CA (US)

(72) Inventors: Max J. Trejo, San Jose, CA (US); Jeffrey M. DiCarlo, Austin, TX (US)

(21) Appl. No.: 18/015,480

(22) PCT Filed: Jul. 19, 2021
(86) PCT No.: PCT/US2021/042225

§ 371 Date: Jan. 10, 2023

Related U.S. Application Data
(60) Provisional application No. 63/054,093, filed on Jul. 20, 2020.

(51) Int. Cl. G06T 7/00

(52) U.S. Cl. CPC G06T 7/0014; G06T 2207/10064

(57) ABSTRACT

An illustrative system may include an imaging device and an image processing system. The imaging device may be configured to output first image information corresponding to a first wavelength band associated with fluorescence emitted by a substance and may be configured to output second image information corresponding to a second wavelength band different from the first wavelength band and associated with the fluorescence emitted by the substance. The image processing system may be configured to receive the first image information and the second image information from the imaging device and may determine, based on a comparison of the first image information to the second image information, a property of the substance.

### RELATED APPLICATIONS

The present application claims priority to U.S. Provisional Pat. Application No. 63/054,093, filed on Jul. 20, 2020, the contents of which are hereby incorporated by reference in their entirety.

### BACKGROUND INFORMATION

Some medical imaging systems are configured to generate fluorescence images of a scene within a body while the body undergoes a medical procedure. The fluorescence images allow medical personnel (e.g., a surgeon) to readily identify cellular activity or structures (e.g., blood vasculature) within the scene during the medical procedure.

To facilitate fluorescence imaging, a fluorescence imaging agent (e.g., a dye, protein, or other substance) configured to fluoresce when exposed to fluorescence excitation illumination may be introduced into a bloodstream or other anatomical feature of the body. A medical imaging system may capture the fluorescence emitted by the fluorescence imaging agent and render a fluorescence image based on the captured fluorescence illumination.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system may include an imaging device and an image processing system. The imaging device may be configured to output first image information corresponding to a first wavelength band associated with fluorescence emitted by a substance and output second image information corresponding to a second wavelength band different from the first wavelength band and associated with the fluorescence emitted by the substance. The image processing system may be configured to receive the first image information and the second image information from the imaging device and determine, based on a comparison of the first image information to the second image information, a property of the substance.

An illustrative apparatus may include one or more processors and memory storing executable instructions that, when executed by the one or more processors, may cause the apparatus to obtain first image information corresponding to a first wavelength band associated with fluorescence emitted by a substance, obtain second image information corresponding to a second wavelength band different from the first wavelength band and associated with the fluorescence emitted by the substance, and determine, based on a comparison of the first image information to the second image information, a property of the substance.

An illustrative non-transitory computer-readable medium may store instructions that, when executed, may cause a processor of a computing device to receive, from an imaging device, first image information corresponding to a first wavelength band associated with fluorescence emitted by a substance; receive, from the imaging device, second image information corresponding to a second wavelength band different from the first wavelength band and associated with the fluorescence emitted by the substance; and determine, based on the first and second image information, a property of the substance.

An illustrative method may include receiving, by an image processing system from an imaging device, a plurality of sets of image information each corresponding to a different wavelength band included in a plurality of wavelength bands associated with fluorescence emitted by a substance; and determining, by the image processing system and based on a comparison of the plurality of sets of image information, a property of the substance.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative medical imaging system.

FIG. 2 shows a graph of spectral intensity plots generated for a fluorescing substance.

FIG. 3 shows an illustrative configuration in which an image processing system is configured to obtain first image information and second image information associated with a fluorescence image captured by an imaging device and generate fluorescing substance property data based on the first and second image information.

FIG. 4 shows wavelength bands for which image information may be obtained by an image processing system to determine a property of a fluorescing substance.

FIG. 5 shows an illustrative configuration in which an image processing system is configured to access relationship data that specifies relationships between different ratios and possible values for a property of a fluorescing substance.

FIG. 6 shows an illustrative implementation of relationship data that may be accessed by an image processing system to determine a concentration of a fluorescing substance.

FIG. 7 shows a configuration in which an image processing system is configured to determine a slope between a characteristic of first image information and a characteristic of second image information.

FIG. 8 shows an illustrative configuration in which an image processing system is configured to provide fluorescing substance property data and fluorescence image data to a display device.

FIG. 9 shows an illustrative user interface.

FIG. 10 shows an illustrative imaging configuration in which an imaging device is configured to output first and second image information used by an imaging processing system to generate fluorescing substance property data.

FIGS. 11-14 shows illustrative implementations of the imaging configuration shown in FIG. 10.

FIGS. 15-16 show illustrative methods.

FIG. 17 shows an illustrative computer-assisted medical system.

FIG. 18 shows an illustrative computing device.

### DETAILED DESCRIPTION

Apparatuses, systems, and methods for image-based determination of a property of a fluorescing substance are described herein. For various reasons, it may be beneficial for a surgeon or other user to be aware of a property of the fluorescing substance (e.g., a concentration level of a fluorescence imaging agent) within a scene at any given time while a fluorescence image of the scene is being displayed by a medical imaging system. However, fluorescence images are often based on signal intensity and therefore affected by many factors, such as illumination, tissue depth, and camera/imager position. As such, it has been difficult or impossible to use the fluorescence image to provide a quantitative and accurate measure of fluorescence imaging agent amounts (e.g., concentration levels and/or total amounts) or other properties of the fluorescence imaging agent.

Apparatuses, systems, and methods described herein may beneficially provide accurate, objective, and/or substantially real-time information representative of amounts (e.g., concentration levels and/or total amounts), identities, and/or other properties of a fluorescing substance within a body during a medical procedure. This may allow a user (e.g., a surgeon) and/or system (e.g., a computer-assisted medical system) to more accurately and effectively determine an amount of time that the substance will fluoresce during a medical procedure compared to conventional scenarios in which accurate representations of such properties are not provided, thereby allowing the user and/or system to make better informed decisions during the medical procedure. Apparatuses, systems, and methods described herein may additionally or alternatively facilitate accurate and effective clinical trials and studies with respect to different fluorescence imaging agents and/or provide additional or alternative advantages and benefits as described herein.

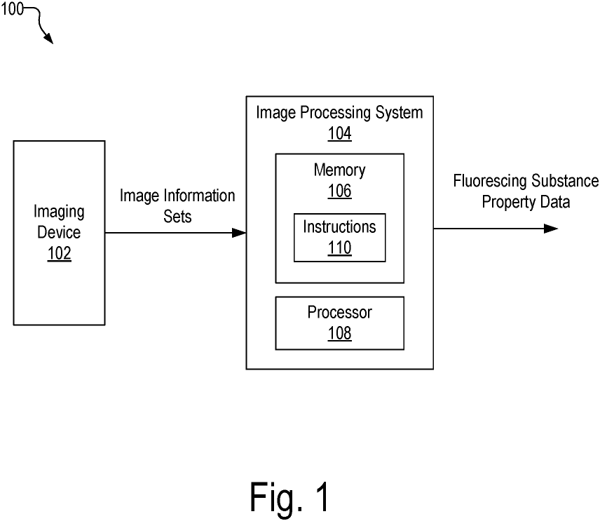

FIG. 1 shows an illustrative medical imaging system 100 configured to generate images of a scene during a medical procedure. In some examples, the scene may include a surgical area associated with a body on or within which the medical procedure is being performed (e.g., a body of a live animal, a human or animal cadaver, a portion of human or animal anatomy, tissue removed from human or animal anatomies, non-tissue work pieces, training models, etc.).

As shown, medical imaging system 100 includes an imaging device 102 in communication with an image processing system 104. Medical imaging system 100 may include additional or alternative components as may serve a particular implementation. In some examples, medical imaging system 100 or certain components of medical imaging system 100 may be implemented by a computer-assisted medical system.

Imaging device 102 may be implemented by an endoscope or other suitable device configured to capture one or more fluorescence images of a scene. In some examples, imaging device 102 may be further configured to capture one or more visible light images of the scene and/or one or more other images in different wavelength bands. The one or more fluorescence images and one or more images in other wavelength bands may be captured concurrently, alternatingly, and/or in any other suitable manner. Illustrative implementations of imaging device 102 are described herein.

As shown, imaging device 102 may be configured to generate and output image information sets. The image information sets may be generated in any suitable manner, examples of which are described herein.

The image information sets may each be representative of a different spectral component of fluorescence emitted by a substance and together may constitute information that may be used to generate and display a fluorescence image representative of the fluorescence. As such, each image information set may correspond to a different wavelength band associated with the fluorescence emitted by the substance.

For example, the image information sets may include a first image information set (or simply “first image information”) corresponding to a first wavelength band associated with fluorescence emitted by a substance and a second image information set (or simply “second image information”) corresponding to a second wavelength band different from the first wavelength band and associated with the fluorescence emitted by the substance. The first image information may be representative of a first spectral component of the fluorescence and may include wavelengths in the first wavelength band, and the second image information may be representative of a second spectral component of the fluorescence and may include wavelengths in the second wavelength band. In some examples, the first and second wavelength bands do not overlap (e.g., by not sharing any common wavelengths). In other examples, the first and second wavelength bands may partially overlap (e.g., by both including some common wavelengths). While two image information sets are referred to in some of the examples described herein, it will be recognized that imaging device 102 may output any number of image information sets as may serve a particular implementation.

Image processing system 104 may be configured to receive the image information sets from imaging device 102 and determine, based on a comparison of the image information sets, a property of the fluorescing substance. Image processing system 104 may output fluorescing substance property data representative of the property, which may be used to present information representative of the property and/or perform one or more other operations as described herein.

The property determined by image processing system 104 may include an amount of the fluorescing substance (e.g., a concentration (also referred to herein as a “concentration level”) of the fluorescing substance or a total amount of the fluorescing substance), an identity (e.g., a name, a classification, and/or a type) of the fluorescing substance, a peak wavelength of the fluorescing substance (e.g., a wavelength at which a spectral peak of a spectral intensity plot for the fluorescing substance occurs), and/or any other property of the fluorescing substance as may serve a particular implementation. In some examples, the property may be time-dependent (e.g., a value of the property changes over time). To illustrate, an amount of a fluorescing substance may change over time as the substance dissipates within a bloodstream.

For illustrative purposes, the examples provided herein show various ways in which a concentration of a fluorescing substance may be determined by image processing system 104. However, the examples provided herein may additionally or alternatively be used to determine any of the other fluorescing substance properties described herein as may serve a particular implementation. For example, embodiments described herein may be used to determine a total amount of fluorescing substance on a surface of tissue being imaged by an imaging device. To illustrate, image processing system 104 may determine a surface area of a surface of tissue being imaged by an imaging device (e.g., by using depth data representative of a distance of the tissue from the imaging device) and use the determined surface area, in combination with the image information sets described herein, to determine a total amount of fluorescing substance on the surface of the tissue. Image processing system 104 may additionally or alternatively determine a total amount of fluorescing substance within tissue by using various size and/or volumetric estimation heuristics for the tissue as may serve a particular implementation.

Image processing system 104 may be implemented by one or more computing devices and/or computer resources (e.g., processors, memory devices, storage devices, etc.) as may serve a particular implementation. As shown, image processing system 104 may include, without limitation, a memory 106 and a processor 108 selectively and communicatively coupled to one another. Memory 106 and processor 108 may each include or be implemented by computer hardware that is configured to store and/or process computer software. Various other components of computer hardware and/or software not explicitly shown in FIG. 1 may also be included within image processing system 104. In some examples, memory 106 and processor 108 may be distributed between multiple devices and/or multiple locations as may serve a particular implementation.

Memory 106 may store and/or otherwise maintain executable data used by processor 108 to perform any of the functionality described herein. For example, memory 106 may store instructions 110 that may be executed by processor 108. Memory 106 may be implemented by one or more memory or storage devices, including any memory or storage devices described herein, that are configured to store data in a transitory or non-transitory manner. Instructions 110 may be executed by processor 108 to cause image processing system 104 to perform any of the functionality described herein. Instructions 110 may be implemented by any suitable application, software, code, and/or other executable data instance. Additionally, memory 106 may also maintain any other data accessed, managed, used, and/or transmitted by processor 108 in a particular implementation.

Processor 108 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 108 (e.g., when processor 108 is directed to perform operations represented by instructions 110 stored in memory 106), image processing system 104 may perform various operations as described herein.

The substance configured to emit fluorescence captured in the form of one or more fluorescence images by imaging device 102 may be of any suitable type. For example, the substance may include an exogenous substance introduced into the body. To illustrate, the substance may include a fluorescence imaging agent (e.g., indocyanine green (ICG) or any other suitable dye, protein, or other substance) configured to be injected or otherwise introduced into the bloodstream or other anatomical feature of the body. Additionally or alternatively, the substance may include an endogenous substance naturally located within the body (e.g., an endogenous fluorophore). The terms “substance” and “fluorescing substance” are used interchangeably herein.

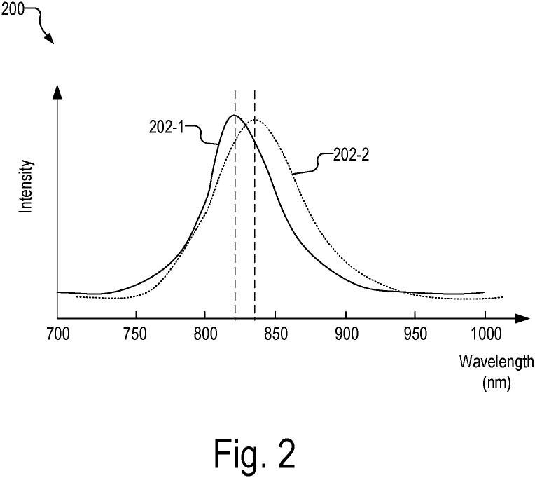

FIG. 2 shows a graph 200 of a first spectral intensity plot 202-1 generated for a fluorescing substance while the fluorescing substance has a first concentration and a second spectral intensity plot 202-2 generated for the same fluorescing substance while the fluorescing substance has a second concentration that is higher than the first concentration.

As shown, the spectral shapes of spectral intensity plots 202-1 and 202-2 are different. For example, spectral intensity plot 202-1 has a sharper rise and fall time than spectral intensity plot 202-2.

Furthermore, as illustrated by the dashed vertical lines shown in FIG. 2, the spectral peaks of spectral intensity plots 202-1 and 202-2 are at different spectral locations (e.g., in terms of wavelength). For example, the spectral peak of spectral intensity plot 202-2 is located at a higher wavelength than the spectral peak of spectral intensity plot 202-1.

The spectral shape and peak differences shown in FIG. 2 are illustrative of the various differences in spectral shapes and peaks that may exist for different concentrations or other properties of a fluorescing substance. Because these differences may be property dependent (e.g., concentration dependent), apparatuses, systems, and methods described herein may utilize image information generated by imaging device 102 to detect one or more characteristics data representative of a spectral intensity plot for a fluorescing substance and thereby identify a property (e.g., a concentration) of the fluorescing substance.

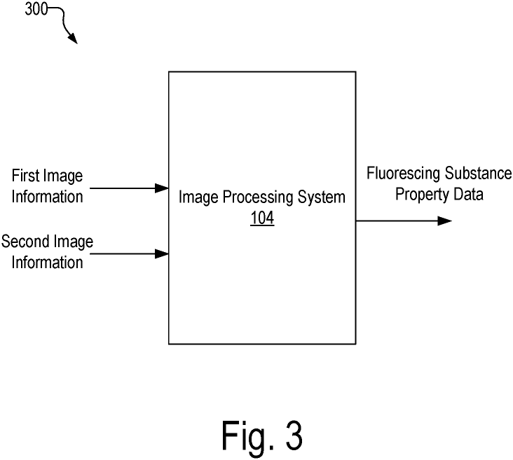

To illustrate, FIG. 3 shows an illustrative configuration 300 in which image processing system 104 is configured to obtain first image information and second image information associated with a fluorescence image captured by imaging device 102 and generate fluorescing substance property data based on the first and second image information.

In this example, the first image information may correspond to a first wavelength band associated with fluorescence emitted by a substance and the second image information may correspond to a second wavelength band different from the first wavelength band and associated with the fluorescence emitted by the substance. For example, the first image information may be representative of a first spectral component of the fluorescence that has wavelengths in the first wavelength band, and the second image information may be representative of a second spectral component of the fluorescence that has wavelengths in the second wavelength band.

Image processing system 104 may obtain the first and second image information in any suitable manner. For example, image processing system 104 may receive the first and second image information from imaging device 102 (e.g., by way of one or more communication interfaces between imaging device 102 and image processing system 104). Additionally or alternatively, image processing system 104 may obtain the first and second image information by generating the first and second image information based on data (e.g., raw image data that has not been fully processed and converted into image information) output by imaging device 102.

As described herein, the spectral shape and spectral peak location for a spectral intensity plot may depend on a property (e.g., a concentration) of a fluorescing substance. As such, a ratio or other comparison between the first and second image information may be unique to a particular property (e.g., a particular concentration) of the fluorescing substance. Accordingly, image processing system 104 may be configured to compare the first image information to the second image information and determine, based on the comparison, a property of the fluorescing substance.

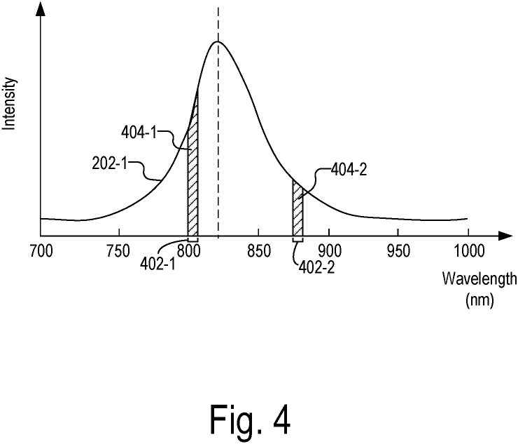

To illustrate, FIG. 4 shows the same spectral intensity plot 202-1 that is shown in FIG. 2. FIG. 4 further depicts a first wavelength band 402-1 and a second wavelength band 402-2 (collectively “wavelength bands 402”) for which image information may be obtained by image processing system 104 to determine a property of the fluorescing substance corresponding to spectral intensity plot 202-1. As shown, each wavelength band 402 may include a plurality of wavelengths that, in this example, are in a near-infrared light region, which includes wavelengths from about 700 nanometers (nm) to about 950 nm and within which the substance emits fluorescence.

Wavelength bands 402 are shown to each be relatively narrow in width (e.g., around 10 nm each), but may be of any suitable width as may serve a particular implementation. Moreover, although each wavelength band 402 in the example of FIG. 4 is located on an opposite side of the spectral peak of spectral intensity plot 202-1, both wavelength bands 402 may be on the same side of the spectral peak in alternative implementations.

In the example of FIG. 4, the first image information obtained by image processing system 104 corresponds to wavelength band 402-1 and is representative of a first spectral component 404-1 of the fluorescence emitted by the substance. Likewise, the second image information obtained by image processing system 104 corresponds to wavelength band 402-2 and is representative of a second spectral component 404-2 of the fluorescence emitted by the substance.

Image processing system 104 may compare the first and second image information to determine a property of the fluorescing substance. This may be performed in any suitable manner.

For example, image processing system 104 may determine a ratio between a characteristic of the first image information and a characteristic of the second image information. Any suitable characteristic of the first and second image information may be compared by image processing system 104 as may serve a particular implementation. For example, in some embodiments, the characteristic of the first image information may include an intensity level corresponding to the first image information, and the characteristic of the second image information may include an intensity level corresponding to the second image information. Image processing system 104 may determine these intensity levels in any suitable manner.

To illustrate, with reference to FIG. 4, image processing system 104 may be configured to determine the intensity level corresponding to the first image information by determining an area of spectral component 404-1. For example, image processing system 104 may integrate data representative of spectral intensity plot 202-1 over wavelength band 402-1. Likewise, image processing system 104 may be configured to determine the intensity level corresponding to the second image information by determining an area of spectral component 404-2. For example, image processing system 104 may integrate data representative of spectral intensity plot 202-1 over wavelength band 402-2.

Additionally or alternatively, image processing system 104 may be configured to determine the intensity level corresponding to the first image information by determining an average intensity level of the first image information within wavelength band 402-1. This may be performed in any suitable manner. For example, image processing system 104 may average each of the intensity levels that define spectral intensity plot 202-1 within wavelength band 402-1. Image processing system 104 may be configured to determine the intensity level corresponding to the second image information in a similar manner.

Additionally or alternatively, image processing system 104 may identify an intensity level within data representative of spectral intensity plot 202-1 that corresponds to a center wavelength within wavelength band 402-1 and designate this intensity level as the intensity level for the first image information. Image processing system 104 may be configured to determine the intensity level corresponding to the second image information in a similar manner.

Once the characteristics of the first and second image information are determined in any suitable manner, image processing system 104 may determine a ratio between (or otherwise compare) the characteristics. Based on this ratio (or comparison), image processing system 104 may determine the property of the fluorescing substance.

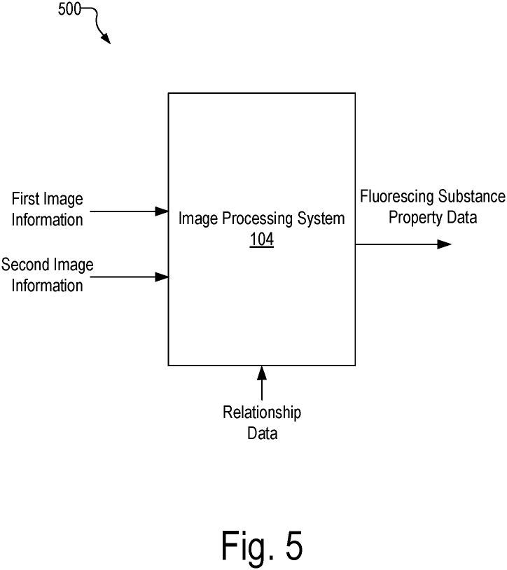

For example, FIG. 5 shows an illustrative configuration 500 in which image processing system 104 is configured to access relationship data that specifies relationships between different ratios and possible values for the property of the fluorescing substance. In configuration 500, image processing system 104 may be configured to determine the property of the fluorescing substance based on the ratio and the relationship data.

The relationship data shown in FIG. 5 may be maintained in memory 106 of image processing system 104 and/or otherwise accessed by image processing system 104. Moreover, the relationship data shown in FIG. 5 may be generated in any suitable manner. For example, the relationship data may be generated by a third party system based on experimental data and provided (e.g., transmitted) to image processing system 104 in any suitable manner.

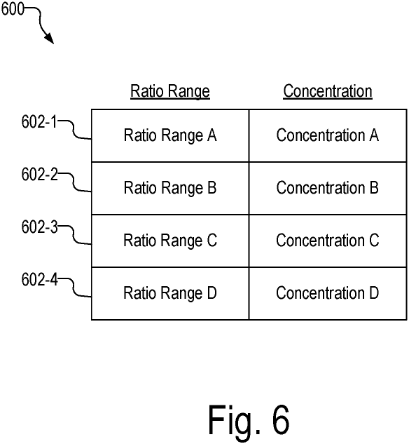

FIG. 6 shows an illustrative implementation of relationship data 600 that may be accessed by image processing system 104 to determine a concentration of the fluorescing substance. As shown, relationship data 600 may be implemented as a table (e.g., a look-up table) that may be stored and accessed in any suitable format. Relationship data 600 may alternatively be implemented in any other suitable format.

As shown, relationship data 600 includes a plurality of entries 602 (e.g., entries 602-1 through 602-4). Each entry 602 specifies a relationship between a particular ratio range and a particular concentration level for a fluorescing substance. For example, entry 602-1 indicates that if a ratio between a characteristic of the first image information and a characteristic of the second image information falls within a range labeled “ratio range A”, image processing system 104 may determine that the concentration of the fluorescing substance has a level of “concentration A”. It will be recognized that relationship data 600 may have any number of entries and that image processing system 104 may use relationship data 600 in any suitable manner to determine a concentration of the fluorescing substance. For example, image processing system 104 may use one or more interpolation and/or other mathematical techniques to determine a specific concentration level for the fluorescing substance based on the determined ratio and relationship data 600.

Image processing system 104 may additionally or alternatively compare the first and second image information to determine the property of the fluorescing substance by determining a slope between a characteristic of the first image information and a characteristic of the second image information. Image processing system 104 may then determine the property of the fluorescing substance based on the slope.

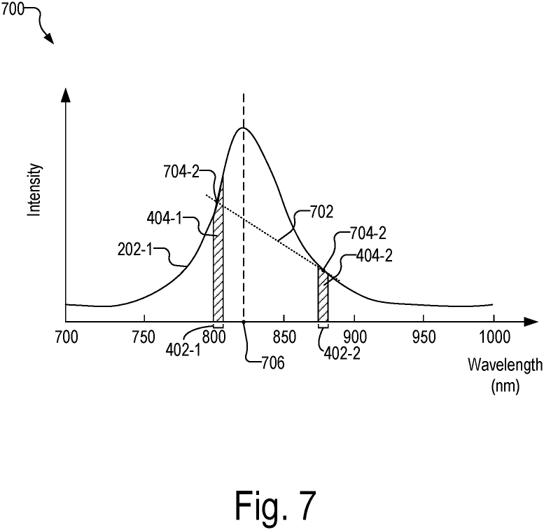

To illustrate, FIG. 7 shows a configuration 700 in which image processing system 104 is configured to determine a slope between a characteristic of the first image information and a characteristic of the second image information. FIG. 7 shows the same spectral intensity plot 202-1 shown in FIG. 4. However, FIG. 7 further depicts a line 702 drawn between an intensity level 704-1 on spectral intensity plot 202-1 that corresponds to the first image information and an intensity level 704-2 on spectral intensity plot 202-1 that corresponds to the second image information. Intensity levels 704-1 and 704-2 (collectively “intensity levels 704”) may be determined in any of the ways described herein.

Based on intensity levels 704, image processing system 104 may determine a slope of line 702, which represents a slope between intensity levels 704. This slope may be unique to a particular property (e.g., concentration) of the fluorescing substance for the reasons described herein. As such, image processing system 104 may determine the property of the fluorescing substance based on the slope. This determination may be further based on relationship data similar to that described in connection with FIGS. 5-6, except that in this example, the relationship data may be slope-based instead of ratio-based.

Image processing system 104 may additionally or alternatively compare the first and second image information to determine the property of the fluorescing substance by determining, based on a comparison of the first image information to the second image information, a wavelength associated with a peak intensity level for the fluorescence. Image processing system 104 may then determine the property of the fluorescing substance based on the determined wavelength.

To illustrate, image processing system 104 may determine intensity levels 704 as described in connection with FIG. 7. Based on these intensity levels 704, image processing system 104 may determine a wavelength associated with a peak of spectral intensity plot 202-1. This wavelength is depicted in FIG. 7 by an indicator 706 and may be determined using any suitable mathematical technique. As the spectral peak location may be unique to a particular property (e.g., concentration) of the fluorescing substance, processing system 104 may determine the property of the fluorescing substance based on the determined wavelength. This determination may be further based on relationship data similar to that described in connection with FIGS. 5-6, except that in this example, the relationship data may be spectral peak-based instead of ratio-based.

While the examples described herein are based on a comparison of first and second image information, it will be recognized that any number of image information sets may be compared one to another to determine a property of a fluorescing substance. For example, image processing system 104 may be configured to compare third image information to the first image information and to the second image information and further base the determination of the property on this comparison.

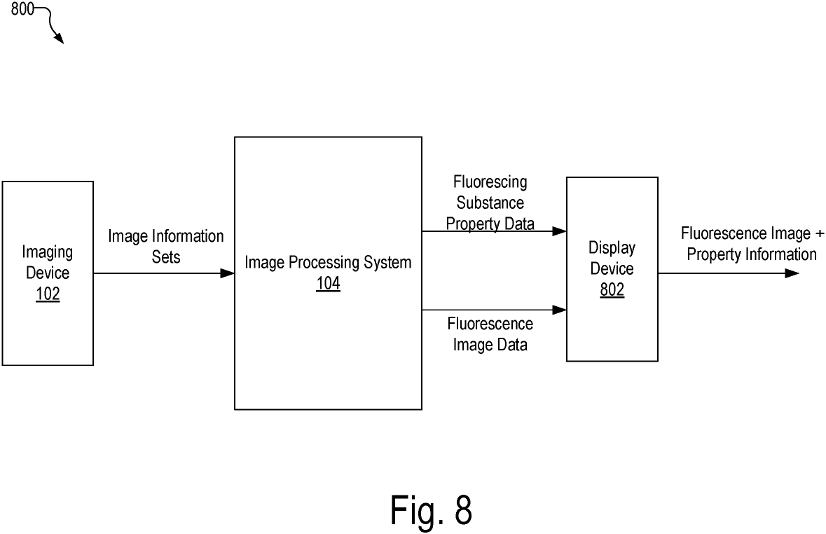

In some examples, image processing system 104 may be configured to present content indicating the property of the fluorescing substance as determined in any of the ways described herein. For example, FIG. 8 shows an illustrative configuration in which image processing system 104 is configured to provide fluorescing substance property data (described herein) and fluorescence image data to a display device 802. The fluorescence image data and fluorescing substance property data may be used by display device 802 to concurrently display a fluorescence image that depicts the fluorescence emitted by the substance and property information representative of the property of the substance. Alternatively, the display device 802 may separately display the fluorescence image and property information (e.g., display at different times), may display the fluorescence image without displaying the property information, or may display the property information without displaying the fluorescence image.

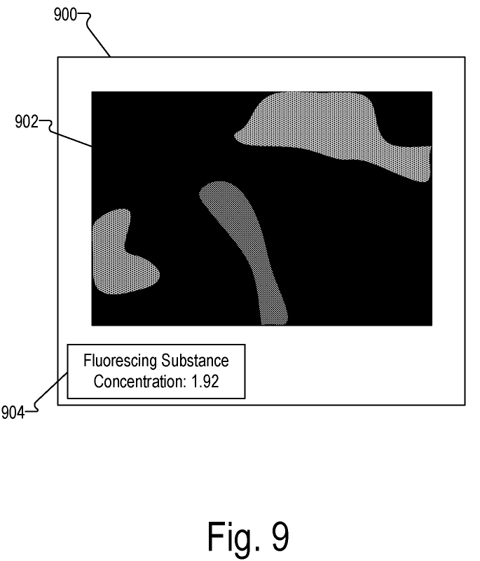

To illustrate, FIG. 9 shows an illustrative user interface 900 that may be displayed by display device 802 as directed by image processing system 104. As shown, user interface 900 includes a fluorescence image 902 and a graphic 904 indicting a concentration level (e.g., a current concentration level) of the substance emitting the fluorescence depicted within fluorescence image 902. Graphic 904 may be updated in substantially real time (or periodically at any suitable time interval) so that a viewer (e.g., a surgeon or other medical personnel) may be aware of the concentration level at any given time. In some examples in which different values of the property of the substance are determined for different pixel regions within an image, as described herein, graphic 904 may include information indicating the different values within the image. For example, image processing system 104 may present first information indicating a first value of the property of the substance for a first pixel region of the image and second information indicating a second value of the property of the substance for a second pixel region of the image. Such information may be presented using numerical values, colors, patterns, and/or any other suitable graphical content.

Imaging device 102 may be configured to generate the image information sets described herein (or data that may be used by image processing system 104 to generate the image information sets) in any suitable manner.

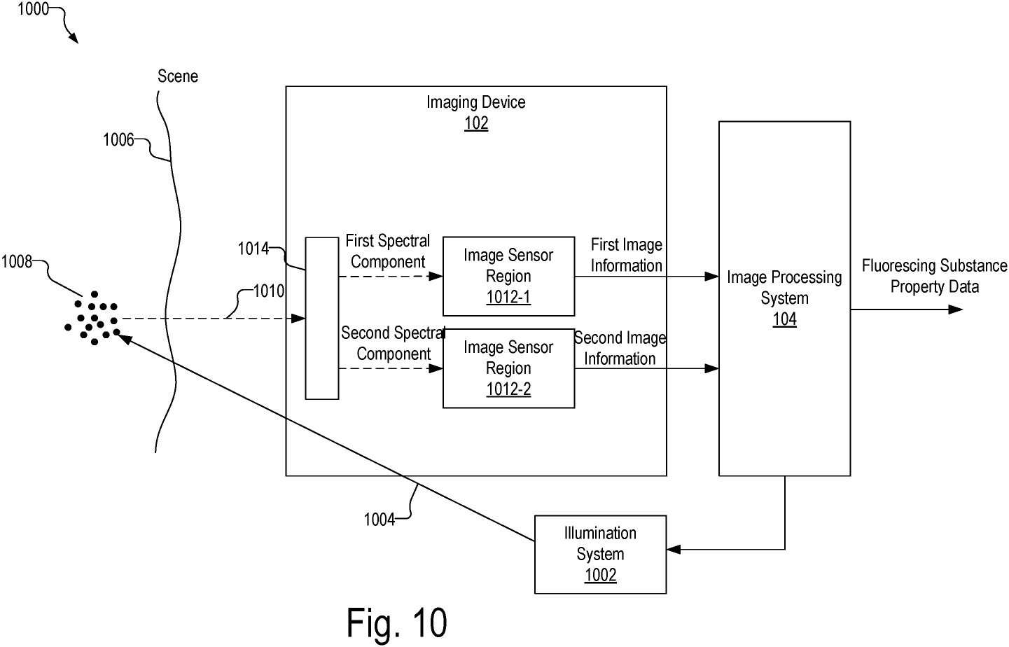

For example, FIG. 10 shows an illustrative imaging configuration 1000 in which imaging device 102 is configured to output first and second image information used by imaging processing system 104 to generate fluorescing substance property data.

As shown, imaging configuration 1000 includes an illumination system 1002 configured to be controlled by image processing system 104. Illumination system 1002 may alternatively be controlled by imaging device 102. Illumination system 1002 may be implemented by one or more illumination sources and may be configured to emit light 1004 (e.g., at the direction of image processing system 104) to illuminate a scene to be imaged by imaging device 102. The light 1004 emitted by illumination system 1002 may include fluorescence excitation illumination (e.g., non-visible light in the near-infrared light region). In some examples, light 1004 may further include visible light.

As shown, light 1004 may travel to the scene through imaging device 102 (e.g., by way of an illumination channel within imaging device 102 that may be implemented by one or more optical fibers, light guides, lenses, etc.). Light 1004 may alternatively travel to the scene by way of an optical path that is outside of imaging device 102.

At least a portion of light 1004 may pass through a surface 1006 (e.g., a surface of or within a body) within the scene and excite a substance 1008 within the scene. Substance 1008 is configured to emit fluorescence 1010 in response to being excited by light 1004 that includes fluorescence excitation illumination.

As shown, imaging device 102 may include a first image sensor region 1012-1 and a second image sensor region 1012-2 (collectively “image sensor regions 1012”). Image sensor region 1012-1 may be configured to detect a first spectral component of fluorescence 1010 and generate the first image information. Image sensor region 1012-2 may be configured to detect a second spectral component of fluorescence 1010. As described herein, image sensor regions 1012 may be implemented by one or more image sensors. Each of the one or more image sensors may be implemented by a charge coupled device (CCD) image sensor, a complementary metal-oxide semiconductor (CMOS) image sensor, a hyperspectral camera, a multispectral camera, and/or any other suitable type of sensing or camera device.

Imaging device 102 may further include a filter stage 1014. Filter stage 1014 may be configured to allow image sensor region 1012-1 to detect the first spectral component of fluorescence 1010 while preventing image sensor region 1012-1 from detecting the second spectral component of fluorescence 1010. Filter stage 1014 may further be configured to allow image sensor region 1012-2 to detect the second spectral component of fluorescence 1010 while preventing image sensor region 1012-2 from detecting the first spectral component of fluorescence 1010. Various implementations of filter stage 1014 are described herein.

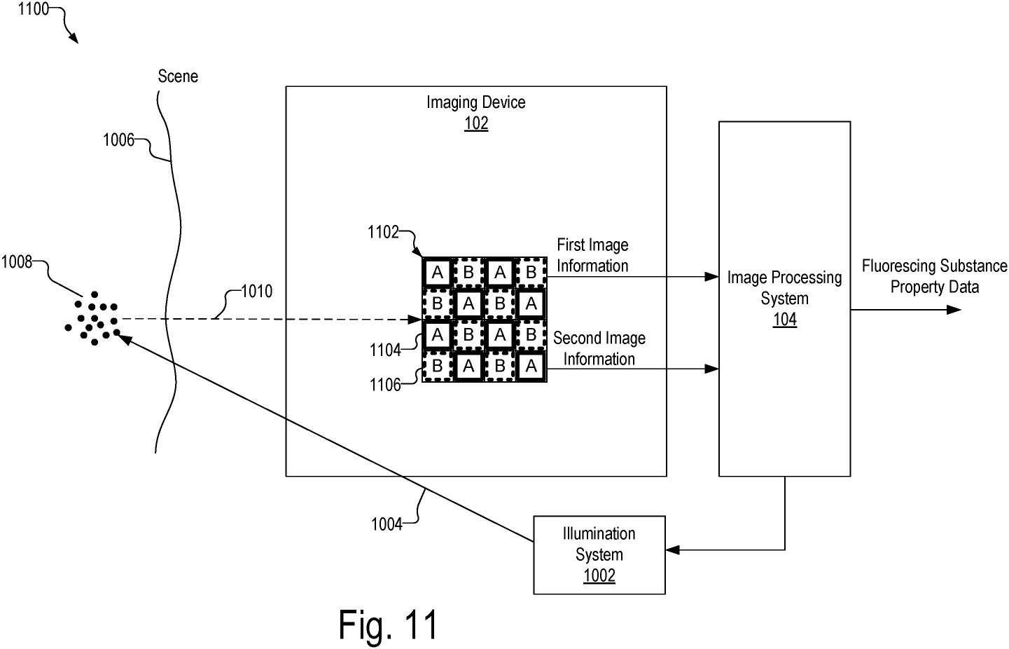

FIG. 11 shows an illustrative implementation 1100 of imaging configuration 1000 in which imaging device 102 includes an image sensor 1102 (e.g., a single image sensor) configured to detect fluorescence 1010. As shown, image sensor 1102 includes a first set of pixels (e.g., pixels labeled “A” in FIG. 11) that implements image sensor region 1012-1 and a second set of pixels (e.g., pixels labeled “B” in FIG. 11) that implements image sensor region 1012-2. Each set of pixels may include any number of pixels in any suitable arrangement on image sensor 1102 as may serve a particular implementation.

In implementation 1100, filter stage 1014 may be implemented by a first filter and a second filter. The first filter may be configured to cover the first set of pixels and is represented in FIG. 11 by a solid box (e.g., box 1104) on top of each of the pixels labeled “A”. In this configuration, the first filter may be configured to prevent the first set of pixels from detecting the second spectral component of fluorescence 1010 (while allowing the first set of pixels to detect the first spectral component of fluorescence 1010).

The second filter may be configured to cover the second set of pixels and is represented in FIG. 11 by a dashed line box (e.g., box 1106) on top of each of the pixels labeled “B”. In this configuration, the second filter may be configured to prevent the second set of pixels from detecting the first spectral component of fluorescence 1010 (while allowing the second set of pixels to detect the second spectral component of fluorescence 1010).

The first and second filters illustrated in FIG. 11 may be implemented by any suitable type of bandpass filter as may serve a particular implementation. For example, the first and second filters may each be implemented by one or more filters (e.g., one or more glass filters) configured to cover (e.g., be on top of) or be integrated into image sensor 1102.

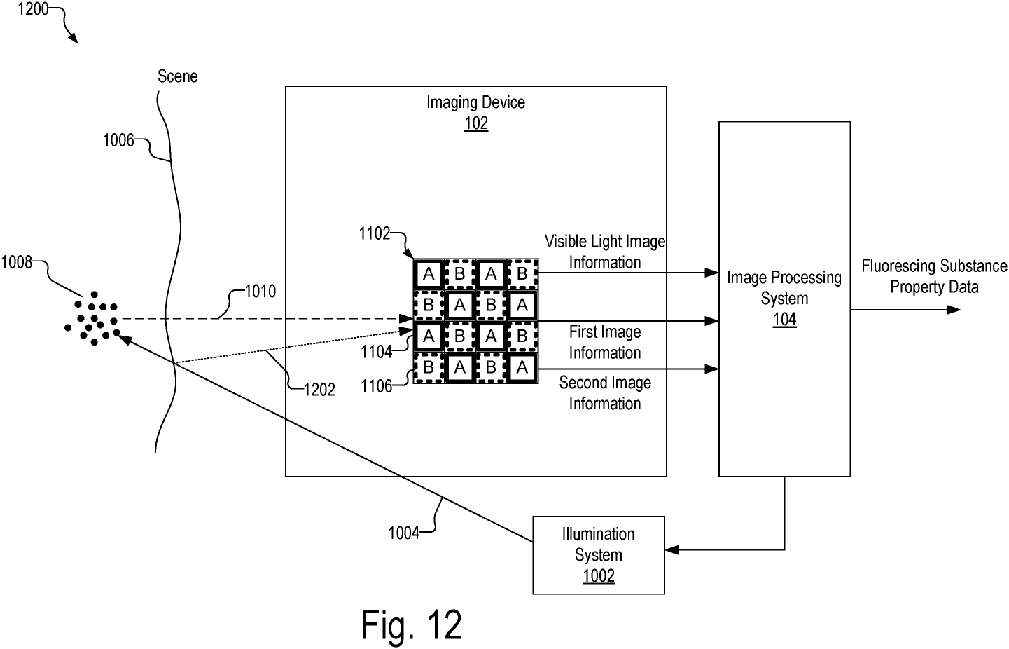

FIG. 12 shows another illustrative implementation 1200 of imaging configuration 1000. In implementation 1200, light 1004 includes both fluorescence excitation illumination and visible light applied either concurrently or sequentially. As shown, a visible light portion 1202 of light 1004 reflects off of surface 1006 and is detected by image sensor 1102,

As described in connection with FIG. 11, image sensor 1102 includes first and second sets of pixels covered by first and second filters configured to allow the first and second sets of pixels to detect the first and second spectral components of fluorescence 1010 and output first and second image information. In implementation 1200, at least some of the pixels of image sensor 1102 may also be configured to detect visible light portion 1202 and output visible light image information representative of a visible light image of surface 1006. To this end, image sensor 1102 may include one or more color filters (e.g., a Bayer filter mosaic and/or any other suitable arrangement of color filters) configured to cover the pixels of image sensor 1102 that are used to detect visible light portion 1202.

In some examples, image processing system 104 may be configured to direct imaging device 102 to alternate between using image sensor 1102 to detect fluorescence 1010 and using image sensor 1102 to detect visible light portion 1202. In this manner, the same pixels may be used to detect fluorescence 1010 and visible light portion 1202.

In alternative examples, image processing system 104 may be configured to direct imaging device 102 to use image sensor 1102 to concurrently detect fluorescence 1010 and visible light portion 1202. In these alternative examples, some pixels of image sensor 1102 may be dedicated to detecting only fluorescence 1010 and other pixels of image sensor 1102 may be dedicated to detecting only visible light portion 1202.

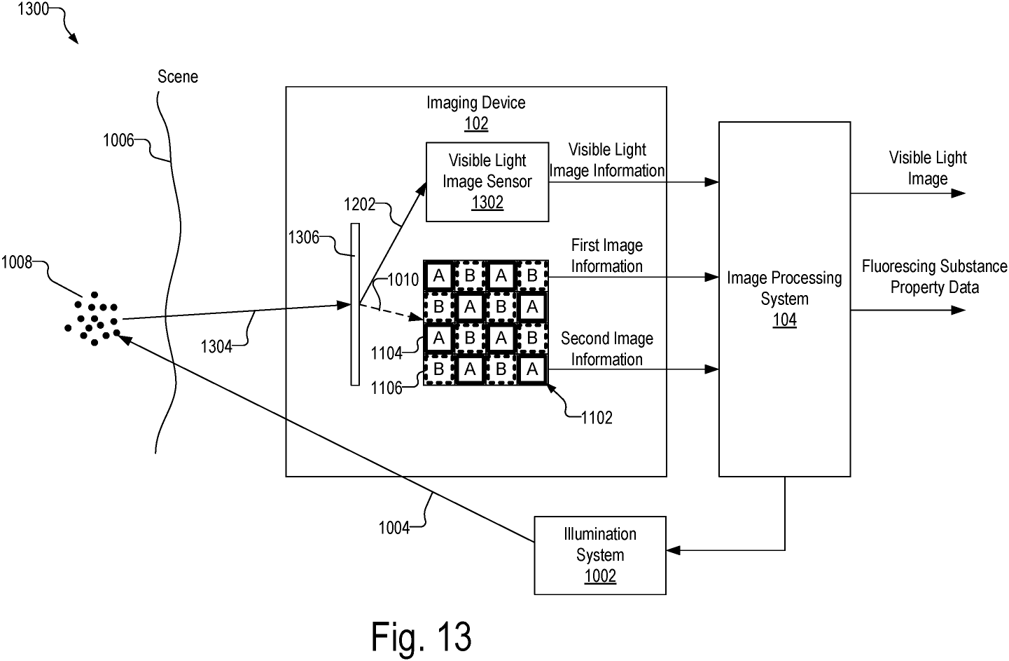

FIG. 13 shows another illustrative implementation 1300 of imaging configuration 1000. In implementation 1300, imaging device 102 includes the image sensor 1102 described in connection with FIG. 11 and a separate visible light image sensor 1302 configured to detect visible light.

In implementation 1300, light 1004 may include both fluorescence excitation illumination and visible light applied either concurrently or sequentially. Combined light 1304, which includes fluorescence 1010 and visible light portion 1202, described above, is received by imaging device 102 and may pass through a splitter 1306 configured to optically direct visible light portion 1202 to visible light image sensor 1302, which outputs visible light image information based on visible light portion 1202. Splitter 1306 may be further configured to optically direct fluorescence 1010 to image sensor 1102, which may output first and second image information as described herein. As such, in implementation 1300, splitter 1306 may be configured to implement filter stage 1014.

Splitter 1306 may be implemented in any suitable manner. For example, splitter 1306 may be implemented by a dichroic mirror (or any other suitable type of mirror or optical splitter) configured and positioned to optically direct visible light portion 1202 to visible light image sensor 1302 (while preventing fluorescence 1010 from being optically directed to visible light image sensor 1302) and optically direct fluorescence 1010 to image sensor 1102 (while preventing visible light portion 1202 from being optically directed to image sensor 1102).

In the implementations shown in FIGS. 11-13, the first and second image information may collectively correspond to an entire pixel area of image sensor 1102. In this manner, the property of the substance may be determined for the entire scene represented by an image captured by image sensor 1102.

In some alternative embodiments, the first and second image information may correspond to a particular pixel region of image sensor 1102 (e.g., a region that is smaller than the entire pixel area of image sensor 1102). For example, image information for only a subset of the pixels shown in FIGS. 11-13 may be used to determine the property of the substance. Such determination may be performed, for example, on a pixel region-by-region basis such that image processing system 104 and/or a user may ascertain substance property differences within tissue depicted in a single image.

By way of example, the concentration level of a fluorescence imaging agent may differ depending on the type of tissue that the fluorescence imaging agent is in. To illustrate, the concentration level of a fluorescence imaging agent may be generally higher in healthy tissue compared to a concentration level of the fluorescence imaging agent in tissue affected by a tumor. By presenting information representative of different concentration levels corresponding to tissue depicted in different pixel regions of a single image, image processing system 104 may allow a user to visually distinguish between healthy tissue and non-healthy tissue.

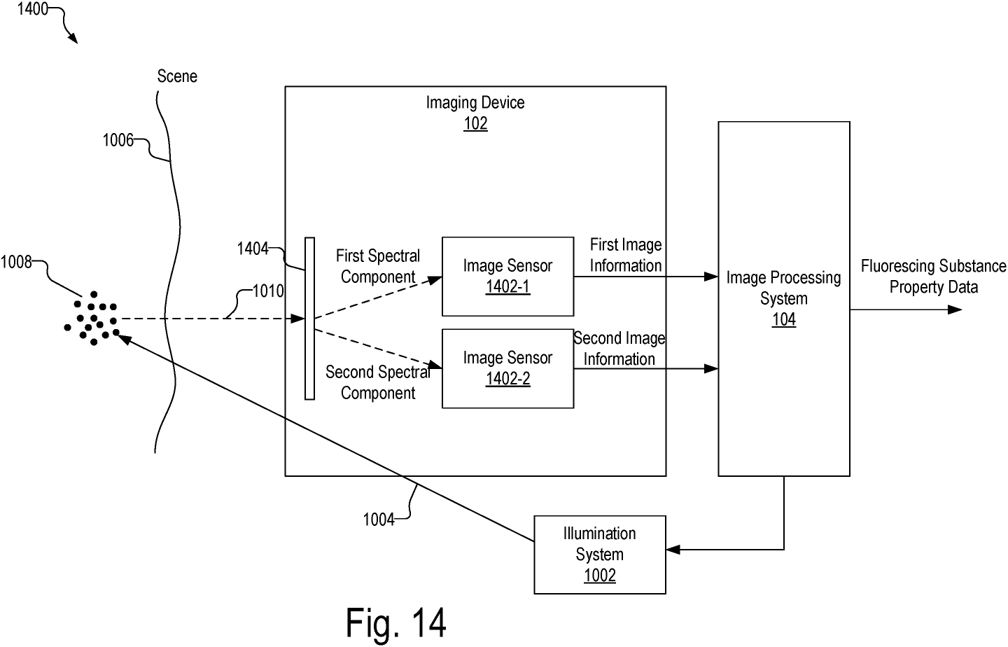

FIG. 14 shows another illustrative implementation 1400 of imaging configuration 1000. In implementation 1400, imaging device 102 includes a first image sensor 1402-1 that implements image sensor region 1012-1 and a second image sensor 1402-2 that implements image sensor region 1012-2. In some examples, image sensor 1402-2 may be physically separate from image sensor 1402-1.

As shown, neither of image sensors 1402 are covered by a filter. Rather, imaging device 102 may include a splitter 1404 configured to optically direct the first spectral component of fluorescence 1010 to image sensor 1402-1 and the second spectral component of fluorescence 1010 to image sensor 1402-2. Image sensors 1402-1 and 1402-2 may accordingly generate the first and second image information, respectively.

Splitter 1404 may be implemented in any suitable manner. For example, splitter 1404 may be implemented by a dichroic mirror (or any other suitable type of mirror or optical splitter) configured and positioned to optically direct the first spectral component to image sensor 1402-1 (while preventing the second spectral component from being optically directed to image sensor 1402-1) and optically direct the second spectral component to image sensor 1402-2 (while preventing the first spectral component from being optically directed to image sensor 1402-2).

Other implementations of imaging configuration 1000 are possible. For example, in an alternative implementation, imaging device 102 may include a single image sensor that implements both image sensor region 1012-1 and image sensor region 1012-2. In this implementation, the single image sensor might not include any filters associated therewith. Rather, the single image sensor may be configured to sequentially output the first image information followed by the second image information. For example, the single image sensor may be configured to sequentially output the first image information followed by the second image information as different wavelength fluorescence excitation illumination signals are applied to the fluorescing substance.

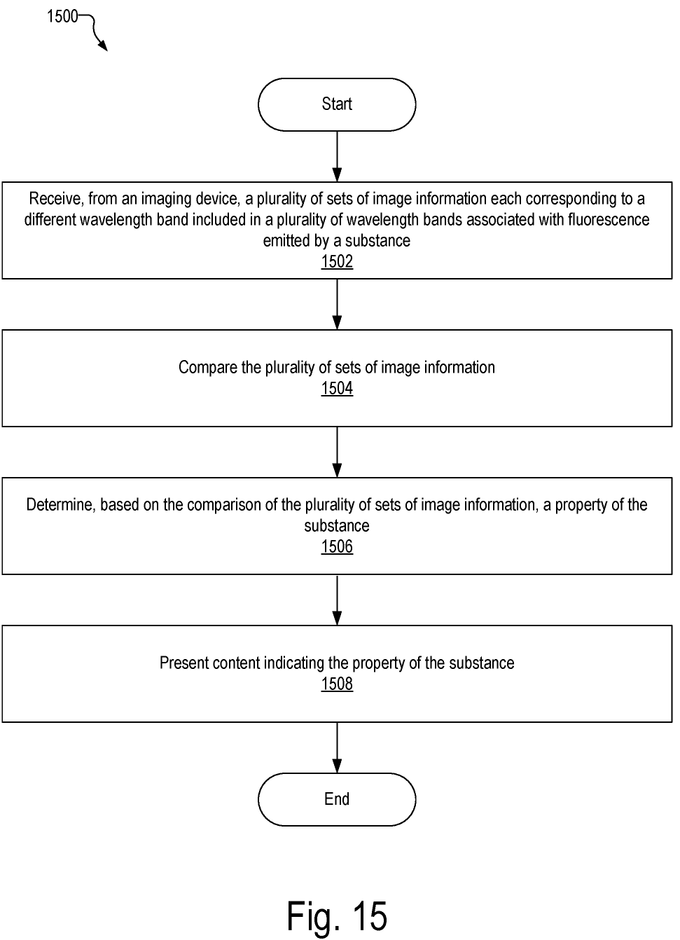

FIG. 15 shows an illustrative method 1500 that may be performed by image processing system 104 and/or any implementation thereof. While FIG. 15 depicts illustrative operations according to one embodiment, other embodiments may omit, add to, reorder, and/or modify any of the operations shown in FIG. 15. Each of the operations shown in FIG. 15 may be performed in any of the ways described herein.

At operation 1502, an image processing system may receive from an imaging device, a plurality of sets of image information each corresponding to a different wavelength band included in a plurality of wavelength bands associated with fluorescence emitted by a substance. For example, the image processing system may receive first image information corresponding to a first wavelength band associated with the fluorescence emitted by the substance and second image information corresponding to a second wavelength band different from the first wavelength band and associated with the fluorescence emitted by the substance. The image processing system may additionally receive any other number of image information corresponding to other wavelength bands associated with the fluorescence as may serve a particular implementation.

At operation 1504, the imaging system may optionally compare the plurality of sets of image information. The comparison may be performed in any of the ways described herein.

At operation 1506, the image processing system may determine, based on the comparison of the plurality of sets of image information, a property of the substance. This may be performed in any of the ways described herein.

For example, based on first and second image information received at operation 1502, the image processing system may determine a ratio between a characteristic of the first image information and a characteristic of the second image information and determine the property of the substance based on the ratio. Additionally or alternatively, the image processing system may access relationship data that specifies relationships between different ratios and the property of the substance and determine the property of the substance based on the ratio and the relationship data.

Additionally or alternatively, based on first and second image information received at operation 1502, the image processing system may determine a slope between a characteristic of the first image information and a characteristic of the second image information and determine the property of the substance based on the slope.

Additionally or alternatively, based on first and second image information received at operation 1502, the image processing system may determine based on the comparison of the first image information to the second image information, a wavelength associated with a peak intensity level for the fluorescence. In these examples, the image processing system may determine the property of the substance based on the wavelength associated with the peak intensity level for the fluorescence.

At operation 1508, the image processing system may optionally present content indicating the property of the substance (e.g., a concentration of the substance, a total amount of the substance, an identity of the substance, a wavelength associated with a peak intensity level for the fluorescence of the substance, etc.). For example, the image processing system may direct a display device to display a graphic indicating the property of the substance together with an image depicting the fluorescence.

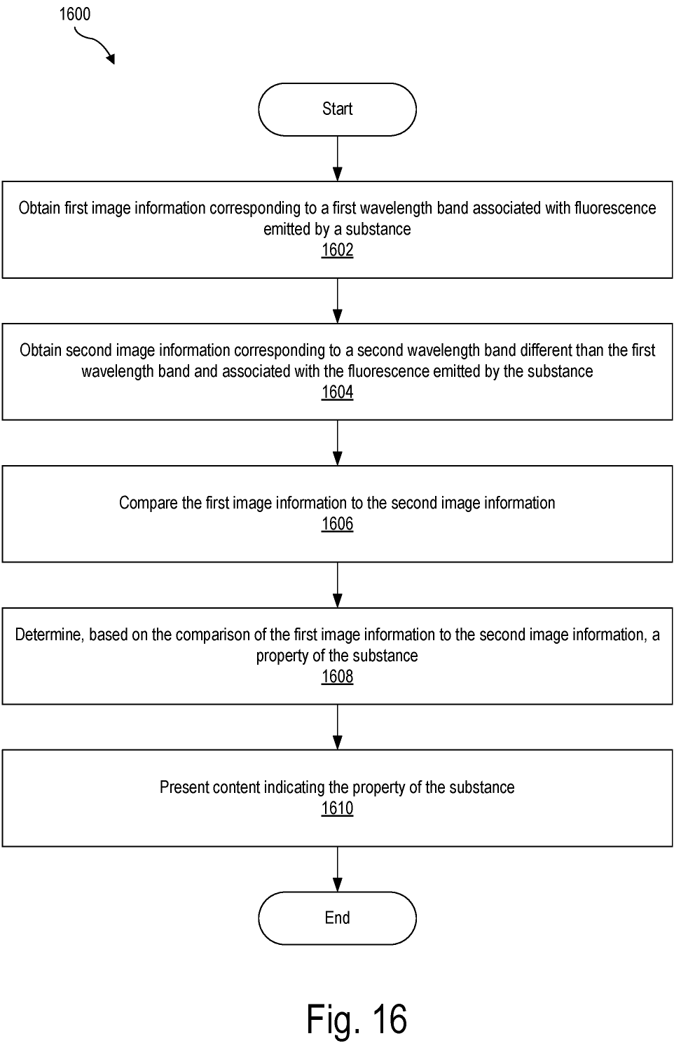

FIG. 16 shows another illustrative method 1600 that may be performed by image processing system 104 and/or any implementation thereof. While FIG. 16 depicts illustrative operations according to one embodiment, other embodiments may omit, add to, reorder, and/or modify any of the operations shown in FIG. 16. Each of the operations shown in FIG. 16 may be performed in any of the ways described herein.

At operation 1602, an image processing system may obtain first image information corresponding to a first wavelength band associated with fluorescence emitted by a substance.

At operation 1604, the image processing system may obtain second image information corresponding to a second wavelength band different from the first wavelength band and associated with the fluorescence emitted by the substance.

In some examples, the imaging processing system may optionally obtain third image information corresponding to a third wavelength band different from the first and second wavelength bands and associated with the fluorescence emitted by the substance and perform the other operations described in connection with FIG. 16 based on the first, second, and third image information. However, for illustrative purposes, the following operations are described in terms of first and second image information.

Operations 1602 and 1604 may be performed in any of the ways described herein. For example, the image processing system may obtain the first and second image information by receiving the first and second image information from one or more imaging devices configured to capture one or more images of the fluorescence. Additionally or alternatively, the image processing system may obtain the first and second image information by generating, based on imaging data transmitted to the image processing system by one or more imaging devices, the first and second image information.

At operation 1606, the image processing system may optionally compare the first image information to the second image information. This comparison may be performed in any of the ways described herein.

At operation 1608, the image processing system may determine, based on the comparison of the first image information to the second image information, a property of the substance. This may be performed in any of the ways described herein.

For example, the image processing system may determine a ratio between a characteristic of the first image information and a characteristic of the second image information and determine the property of the substance based on the ratio. Additionally or alternatively, the image processing system may access relationship data that specifies relationships between different ratios and the property of the substance and determine the property of the substance based on the ratio and the relationship data.

Additionally or alternatively, the image processing system may determine a slope between a characteristic of the first image information and a characteristic of the second image information and determine the property of the substance based on the slope.

Additionally or alternatively, the image processing system may determine, based on the comparison of the first image information to the second image information, a wavelength associated with a peak intensity level for the fluorescence. In these examples, the image processing system may determine the property of the substance based on the wavelength associated with the peak intensity level for the fluorescence.

At operation 1610, the image processing system may optionally present content indicating the property of the substance (e.g., a concentration of the substance, a total amount of the substance, an identity of the substance, a wavelength associated with a peak intensity level for the fluorescence of the substance, etc.). For example, the image processing system may direct a display device to display a graphic indicating the property of the substance together with an image depicting the fluorescence.

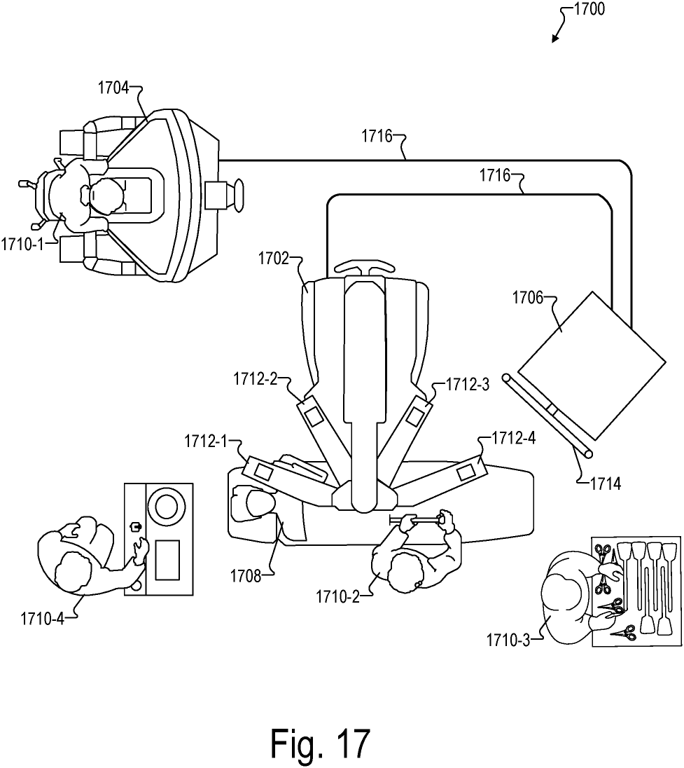

Imaging device 102 and/or image processing system 104 may be implemented by, included in, and/or otherwise associated with a computer-assisted medical system used to perform a medical procedure (e.g., a fluorescence-guided medical procedure) on a body. FIG. 17 shows an illustrative computer-assisted medical system 1700 that may be used to perform various types of medical procedures including surgical and/or non-surgical procedures.

As shown, computer-assisted medical system 1700 may include a manipulator assembly 1702 (a manipulator cart is shown in FIG. 17), a user control apparatus 1704, and an auxiliary apparatus 1706, all of which are communicatively coupled to each other. Computer-assisted medical system 1700 may be utilized by a medical team to perform a computer-assisted medical procedure or other similar operation on a body of a patient 1708 or on any other body as may serve a particular implementation. As shown, the medical team may include a first user 1710-1 (such as a surgeon for a surgical procedure), a second user 1710-2 (such as a patient-side assistant), a third user 1710-3 (such as another assistant, a nurse, a trainee, etc.), and a fourth user 1710-4 (such as an anesthesiologist for a surgical procedure), all of whom may be collectively referred to as “users 1710,” and each of whom may control, interact with, or otherwise be a user of computer-assisted medical system 1700. More, fewer, or alternative users may be present during a medical procedure as may serve a particular implementation. For example, team composition for different medical procedures, or for non-medical procedures, may differ and include users with different roles.

While FIG. 17 illustrates an ongoing minimally invasive medical procedure such as a minimally invasive surgical procedure, it will be understood that computer-assisted medical system 1700 may similarly be used to perform open medical procedures or other types of operations. For example, operations such as exploratory imaging operations, mock medical procedures used for training purposes, and/or other operations may also be performed.

As shown in FIG. 17, manipulator assembly 1702 may include one or more manipulator arms 1712 (e.g., manipulator arms 1712-1 through 1712-4) to which one or more instruments may be coupled. The instruments may be used for a computer-assisted medical procedure on patient 1708 (e.g., in a surgical example, by being at least partially inserted into patient 1708 and manipulated within patient 1708). While manipulator assembly 1702 is depicted and described herein as including four manipulator arms 1712, it will be recognized that manipulator assembly 1702 may include a single manipulator arm 1712 or any other number of manipulator arms as may serve a particular implementation. While the example of FIG. 17 illustrates manipulator arms 1712 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 1712 shown in FIG. 17.

During the medical operation, user control apparatus 1704 may be configured to facilitate teleoperational control by user 1710-1 of manipulator arms 1712 and instruments attached to manipulator arms 1712. To this end, user control apparatus 1704 may provide user 1710-1 with imagery of an operational area associated with patient 1708 as captured by an imaging device. To facilitate control of instruments, user control apparatus 1704 may include a set of master controls. These master controls may be manipulated by user 1710-1 to control movement of the manipulator arms 1712 or any instruments coupled to manipulator arms 1712.

Auxiliary apparatus 1706 may include one or more computing devices configured to perform auxiliary functions in support of the medical procedure, such as providing insufflation, electrocautery energy, illumination or other energy for imaging devices, image processing, or coordinating components of computer-assisted medical system 1700. In some examples, auxiliary apparatus 1706 may be configured with a display monitor 1714 configured to display one or more user interfaces, or graphical or textual information in support of the medical procedure. In some instances, display monitor 1714 may be implemented by a touchscreen display and provide user input functionality. Augmented content provided by a region-based augmentation system may be similar, or differ from, content associated with display monitor 1714 or one or more display devices in the operation area (not shown).

Manipulator assembly 1702, user control apparatus 1704, and auxiliary apparatus 1706 may be communicatively coupled one to another in any suitable manner. For example, as shown in FIG. 17, manipulator assembly 1702, user control apparatus 1704, and auxiliary apparatus 1706 may be communicatively coupled by way of control lines 1716, which may represent any wired or wireless communication link as may serve a particular implementation. To this end, manipulator assembly 1702, user control apparatus 1704, and auxiliary apparatus 1706 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

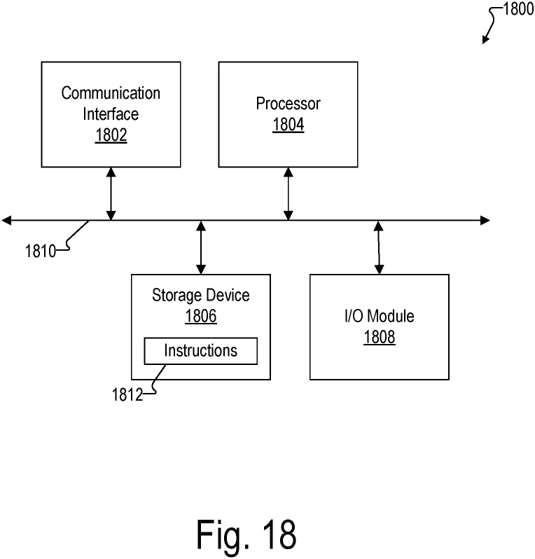

FIG. 18 shows an illustrative computing device 1800 that may be specifically configured to perform one or more of the processes described herein. Any of the systems, computing devices, and/or other components described herein may be implemented by computing device 1800.

As shown in FIG. 18, computing device 1800 may include a communication interface 1802, a processor 1804, a storage device 1806, and an input/output (“I/O”) module 1808 communicatively connected one to another via a communication infrastructure 1810. While an illustrative computing device 1800 is shown in FIG. 18, the components illustrated in FIG. 18 are not intended to be limiting. Additional or alternative components may be used in other embodiments. Components of computing device 1800 shown in FIG. 18 will now be described in additional detail.

Communication interface 1802 may be configured to communicate with one or more computing devices. Examples of communication interface 1802 include, without limitation, a wired network interface (such as a network interface card), a wireless network interface (such as a wireless network interface card), a modem, an audio/video connection, and any other suitable interface.

Processor 1804 generally represents any type or form of processing unit capable of processing data and/or interpreting, executing, and/or directing execution of one or more of the instructions, processes, and/or operations described herein. Processor 1804 may perform operations by executing computer-executable instructions 1812 (e.g., an application, software, code, and/or other executable data instance) stored in storage device 1806.

Storage device 1806 may include one or more data storage media, devices, or configurations and may employ any type, form, and combination of data storage media and/or device. For example, storage device 1806 may include, but is not limited to, any combination of the non-volatile media and/or volatile media described herein. Electronic data, including data described herein, may be temporarily and/or permanently stored in storage device 1806. For example, data representative of computer-executable instructions 1812 configured to direct processor 1804 to perform any of the operations described herein may be stored within storage device 1806. In some examples, data may be arranged in one or more databases residing within storage device 1806.

I/O module 1808 may include one or more I/O modules configured to receive user input and provide user output. I/O module 1808 may include any hardware, firmware, software, or combination thereof supportive of input and output capabilities. For example, I/O module 1808 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1808 is configured to provide graphical data to a display for presentation to a user. The graphical data may be representative of one or more graphical user interfaces and/or any other graphical content as may serve a particular implementation.

Advantages and features of the present disclosure can be further described by the following statements.

1. A non-transitory computer-readable medium storing instructions that, when executed, cause a processor of a computing device to: receive, from an imaging device, first image information corresponding to a first wavelength band associated with fluorescence emitted by a substance; receive, from the imaging device, second image information corresponding to a second wavelength band different from the first wavelength band and associated with the fluorescence emitted by the substance; and determine, based on the first and second image information, a property of the substance.

2. The non-transitory computer-readable medium of the preceding statement, wherein the determining the property of the substance comprises: determining a ratio between a characteristic of the first image information and a characteristic of the second image information; and determining the property of the substance based on the ratio.

3. The non-transitory computer-readable medium of any of the preceding statements, wherein: the instructions, when executed, cause the processor of the computing device to access relationship data that specifies relationships between different ratios and the property of the substance; and the determining the property of the substance comprises determining the property of the substance based on the ratio and the relationship data.

4. The non-transitory computer-readable medium of any of the preceding statements, wherein the determining the property of the substance comprises: determining a slope between a characteristic of the first image information and a characteristic of the second image information; and determining the property of the substance based on the slope.

5. The non-transitory computer-readable medium of any of the preceding statements, wherein the property of the substance comprises an amount of the substance.

6. The non-transitory computer-readable medium of any of the preceding statements, wherein the amount comprises one or more of a concentration of the substance or a total amount of the substance.

7. The non-transitory computer-readable medium of any of the preceding statements, wherein the property of the substance comprises an identity of the substance.

8. The non-transitory computer-readable medium of any of the preceding statements, wherein the property of the substance comprises a wavelength associated with a peak intensity level for the fluorescence.

9. The non-transitory computer-readable medium of any of the preceding statements, wherein the instructions, when executed, cause the processor of the computing device to present content indicating the property of the substance.

10, The non-transitory computer-readable medium of any of the preceding statements, wherein the presenting the content comprises directing a display device to display a graphic indicating the property of the substance together with an image depicting the fluorescence.

11. A method comprising: receiving, by an image processing system and from an imaging device, a plurality of sets of image information each corresponding to a different wavelength band included in a plurality of wavelength bands associated with fluorescence emitted by a substance; and determining, by the image processing system and based on a comparison of the plurality of sets of image information, a property of the substance.

12. The method of any of the preceding method statements, wherein the receiving the plurality of sets of image information comprises: receiving first image information corresponding to a first wavelength band associated with the fluorescence emitted by the substance; and receiving second image information corresponding to a second wavelength band different from the first wavelength band and associated with the fluorescence emitted by the substance.

13. The method of any of the preceding method statements, wherein the determining the property of the substance comprises: determining a ratio between a characteristic of the first image information and a characteristic of the second image information; and determining the property of the substance based on the ratio.

14. The method of any of the preceding method statements, further comprising: accessing, by the image processing system, relationship data that specifies relationships between different ratios and the property of the substance; and the determining the property of the substance comprises determining the property of the substance based on the ratio and the relationship data.

15. The method of any of the preceding method statements, wherein the determining the property of the substance comprises: determining a slope between a characteristic of the first image information and a characteristic of the second image information; and determining the property of the substance based on the slope.

16. The method of any of the preceding method statements, wherein the determining the property of the substance comprises: determining, based on the comparison of the first image information to the second image information, a wavelength associated with a peak intensity level for the fluorescence; and determining the property of the substance based on the wavelength associated with the peak intensity level for the fluorescence.

17. The method of any of the preceding method statements, further comprising presenting, by the image processing system, content indicating the property of the substance.

18. The method of any of the preceding method statements, wherein the presenting the content comprises directing a display device to display a graphic indicating the property of the substance together with an image depicting the fluorescence.

In the preceding description, various illustrative embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
an imaging device configured to:
output first image information corresponding to a first wavelength band associated with fluorescence emitted by a substance; and
output second image information corresponding to a second wavelength band different from the first wavelength band and associated with the fluorescence emitted by the substance; and
an image processing system configured to:
receive the first image information and the second image information from the imaging device; and
determine, based on a comparison of the first image information to the second image information, a property of the substance.

2. The system of claim 1, wherein the image processing system is configured to determine the property of the substance by:
determining a ratio between a characteristic of the first image information and a characteristic of the second image information; and
determining the property of the substance based on the ratio.

3. The system of claim 2, wherein the image processing system is further configured to:
access relationship data that specifies relationships between different ratios and possible values for the property of the substance; and
wherein determining the property of the substance based on the ratio comprises determining the property of the substance based on the ratio and the relationship data.

4. The system of claim 3, wherein the image processing system is configured to maintain the relationship data in a memory of the image processing system.

5. The system of claim 2, wherein the characteristic of the first image information comprises an intensity level corresponding to the first image information and the characteristic of the second image information comprises an intensity level corresponding to the second image information.

6. The system of claim 1, wherein the image processing system is configured to determine the property of the substance by:
determining a slope between a characteristic of the first image information and a characteristic of the second image information; and
determining the property of the substance based on the slope.

7. The system of claim 1, wherein the image processing system is configured to determine the property of the substance by:
determining, based on the comparison of the first image information to the second image information, a wavelength associated with a peak intensity level for the fluorescence; and
determining the property of the substance based on the wavelength associated with the peak intensity level for the fluorescence.

8. The system of claim 1, wherein:
the substance is located in a body while the substance emits the fluorescence; and
the substance comprises an exogenous substance introduced into the body.

9. The system of claim 1, wherein:
the substance is located in a body while the substance emits the fluorescence; and
the substance comprises an endogenous substance naturally located within the body.

10. The system of claim 1, wherein the property of the substance comprises an amount of the substance.

11. The system of claim 10, wherein the amount comprises one or more of a concentration of the substance or a total amount of the substance.

12. The system of claim 1, wherein the property of the substance comprises an identity of the substance.

13. The system of claim 1, wherein the property of the substance comprises a wavelength associated with a peak intensity level for the fluorescence.

14. The system of claim 1, wherein the image processing system is further configured to present content indicating the property of the substance.

15-29. (canceled)

30. An apparatus comprising:
one or more processors; and
memory storing executable instructions that, when executed by the one or more processors, cause the apparatus to:
obtain first image information corresponding to a first wavelength band associated with fluorescence emitted by a substance;
obtain second image information corresponding to a second wavelength band different from the first wavelength band and associated with the fluorescence emitted by the substance; and
determine, based on a comparison of the first image information to the second image information, a property of the substance.

31. The apparatus of claim 30, wherein the determining the property of the substance comprises:
determining a ratio between a characteristic of the first image information and a characteristic of the second image information; and
determining the property of the substance based on the ratio.

32. The apparatus of claim 31, wherein:
the instructions, when executed by the one or more processors, further cause the apparatus to access relationship data that specifies relationships between different ratios and the property of the substance; and
the determining the property of the substance comprises determining the property of the substance based on the ratio and the relationship data.

33. The apparatus of claim 32, wherein the memory is configured to store the relationship data.

34. The apparatus of claim 31, wherein the characteristic of the first image information comprises an intensity level corresponding to the first image information and the characteristic of the second image information comprises an intensity level corresponding to the second image information.

35. The apparatus of claim 30, wherein the determining the property of the substance comprises:
determining a slope between a characteristic of the first image information and a characteristic of the second image information; and
determining the property of the substance based on the slope.

36-49. (canceled)
